(19) United States
(12) Patent Application Publication
(10) Pub. No.: US 2002/0165549 A1
(43) Pub. Date: Nov. 7, 2002

(54) Surgical instrument and attachment

(75) Inventors: Samuel Owusu-Akyaw, Southlake, TX (US); Rob Ellins, Euless, TX (US); John K. Henderson, Flower Mound, TX (US); E. Paul Strauss, Grapevine, TX (US); Keith Williams, Memphis, TN (US); Raymond Lyle Schenk, III, Chanhassen, MN (US); Allen P. Hilton, Arlington, TX (US); Brian Highley, Keller, TX (US); Michael John Busker, Oakdale, MN (US); Steven John Lundeen, Ramsey, MN (US); Mark A. Serre, Burns Township, MN (US); Curtis Wayne Quiring, Anoka, MN (US); Bryan Michael Clem, Mound, MN (US); Richard Dennis Wilson, St. Francis, MN (US)

Correspondence Address: HAYNES AND BOONE, LLP, 901 MAIN STREET, SUITE 3100, DALLAS, TX, 75202, US

(73) Assignee: Medtronic, Inc., Minneapolis, MN (US)

(21) Appl. No.: 10/135,608

(22) Filed: Apr. 29, 2002

Related U.S. Application Data
(60) Provisional application No. 60/360,332, filed on Feb. 26, 2002.
(60) Provisional application No. 60/287,456, filed on Apr. 30, 2001.
(60) Provisional application No. 60/352,609, filed on Jan. 28, 2002.

(51) Int. Cl. A61B 17/00

(52) U.S. Cl. 606/80

(57) ABSTRACT

For use in a hospital or other medical facility, provided is a powered surgical instrument. The surgical instrument can be used by doctors and the like for the dissection of bone and tissue. The surgical instrument includes a disposable drive unit and a dissection tool. The drive unit is made of a moldable or non-machined material, such as plastic, which does not have to sustain repeated use and sterilization over an extended period of time.

### CROSS REFERENCE

This invention claims the benefit of U.S. patent Ser. No. 60/360,332 filed Feb. 26, 2002 and U.S. patent Ser. No. 60/287,456 filed Apr. 30, 2001, both of which are hereby incorporated by reference as if reproduced in their entirety.

The following related patent applications are also hereby made of record and incorporated by reference: U.S. patent Ser. No. 10/102,762, U.S. patent Ser. No. 09/303,781, and U.S. patent Ser. No. 60/352,609.

### FIELD OF THE INVENTION

The present invention generally relates to surgical istruments and their use. More particularly, the present invention relates to powered surgical instruments for use in the dissection of bone and other tissue.

### BACKGROUND

Surgical instruments, in general, have several very unique requirements. One requirement is that the instrument must maintain a sterile environment. The instrument must not introduce infections, toxic debris, and other contaminants into a surgical procedure. Typically, a surgical instrument is sterilized before and/or after the surgical procedure using an autoclave to disinfect the instrument and remove any toxic debris and other contaminants.

Additional requirements exist with powered surgical instruments. It is well known in the art to power various types of powered surgical instruments with a drive unit such as a pneumatic or electric motor. For example, various surgical procedures employ rotary-type surgical instruments to dissect bone or other tissue. In their most basic form, such rotary-type surgical instruments include a motor that drives a rotary shaft. The motor is often required to operate at a very high speed and/or torque, such as at speeds between 70,000 to 80,000 revolutions per minute (RPM). In one application, a dissection tool, or “bur,” having a cutting element is driven by the rotary shaft. Typically, an attachment surrounds and supports the dissection tool as it extends from the motor. A collet or coupling arrangement connects the dissection tool to a spindle of the rotary shaft.

Reusable surgical instruments have been designed to satisfy certain levels of reliability over an extended period of time involving several hundred surgeries. Such reliability can be especially difficult and expensive to manufacture and maintain in light of the high speed operation of the powered instrument and the repeated sterilization procedures performed thereon. Furthermore, cleaning and maintenance must be performed to keep the surgical instrument within satisfactory operating parameters.

### SUMMARY

The present disclosure provides many technological advances that can be used, either alone or in combination, to provide an improved powered surgical instrument and/or an improved system and method for using powered surgical instruments.

In one embodiment, the present invention provides a powered surgical drive unit and/or a attachment for a powered surgical drive unit. In one embodiment, the drive unit and/or attachment is not repeatedly used and re-sterilized, and can therefore be made of materials not normally available to surgical instruments. For example, because the drive unit will not be subject to a high temperature autoclave sterilization process (much less repeated sterilization processes), the drive unit can include components that are made of thermoplastic, thermosets, composites, brass, aluminum, magnesium, or zinc, and that are manufactured by die casting, investment casting, injection molding, or metal injection molding. Also, because the drive unit may only be used for a single surgical procedure, manufacturing tolerances can be somewhat relaxed, which further facilitates the use of the above-described components and processes. In view of the advantages offered by these materials and manufacturing techniques associated therewith, the cost of such drive units and/or attachments is significantly less that currently available re-useable drive units and attachments.

In some embodiments, the powered surgical drive unit and/or attachment may purposely include one or more failure points so that they cannot be reused. This can be important to prevent the improper reuse of the device(s) for subsequent surgical procedures. For example, the drive unit can include a component that adversely reacts to a high-temperature autoclave or other sterilization process. The component may include, for further example, a wax or other material that melts or deforms during autoclave and causes the drive unit to be rendered inoperable. As a further example, for an electric motor, a fuse may be included in an electrical path to the motor. The fuse can be of the type that is destroyed during autoclave or other sterilization process, and causes the instrument to be rendered inoperable. Still further, a warning label activated by exposure to a sterilization process may be incorporated into the drive unit and/or attachment to warn user's of potential damage.

Alternatively or in addition, the drive unit may include a single-use connection point. In some embodiments, a single-use coupling connects a fluid hose with a pneumatically powered motor. The single-use coupling includes a first portion and a second portion. The first portion is carried by the pneumatically powered motor. The second portion is carried by the fluid hose. The first and second portions connect to define a fluid path between the pneumatically powered motor and the fluid hose. One of the first and second portions includes at least one retainer for securing the first portion to the second portion. The other of the first and second portions includes a cutting member for destruction of the at least one retainer upon decoupling of the first and second portions. In other embodiments, the fluid hose is permanently secured to the motor. The permanent hose can be constructed of less durable materials because of its disposability.

In some embodiments, the surgical drive unit and/or attachment may not require any type of liquid lubrication. Such embodiments may include components formed of or coated with materials having a low coefficient of friction. In other embodiments, an internal lubrication system can be provided so that external lubrication does not need to be provided. In one example, a self-contained, self-metering, and self-initializing system can be employed. This example system may include a membrane for carrying a predetermined amount of lubrication (e.g., to support approximately 4-8 hours of operation). The lubrication can be activated at an initial operation phase. For example, a high pressure fluid that is used by a pneumatic motor can also serve to puncture the membrane. In another example, some of the materials used in the instrument can degrade, and through degradation, provide lubrication to the remaining portions of the instrument. In yet another example, porous bearing assemblies (e.g., ball bearing assemblies that use powdered or sintered metallurgy technologies) that are impregnated with lubricant can be used.

In some embodiments, all or part of the surgical instrument is constructed of non-magnetic material. In other embodiments, the surgical instrument is constructed entirely or substantially of non-electrical conductive material. Such embodiments can be beneficial for certain surgical procedures, such as those that utilize magnetic resonance imaging.

In one particular form, the present invention provides a kit for a surgical procedure. In one embodiment, the kit includes a drive unit and/or attachment and a dissection tool to be driven by the drive unit. The kit further includes a sealed package enveloping the drive unit and/or attachment, and the dissection tool. The sealed package maintains sterility of the included items prior to the surgical procedure.

In another embodiment, the kit may include a plurality of dissection tools and/or attachments necessary to perform a complete surgical procedure. Such combinations may include, but are not limited to, providing separate kits with components suitable for craniotomy, maxillofacial surgery, spinal surgery, hip and knee surgery, dental procedures and soft tissue resection. The kit simplifies accounting and inventory procedures because the contents of the kit can be simply discarded after use. Furthermore, because the kit is single-use, the cost for the kit can be charged to the patient, and does not need to be considered an expense to be shared across many different patients. Further still, the components inside the kit can be stored with a very high level of sterility, provided by the supplier of the kit.

In another form, the present invention is directed to a surgical technique. The surgical technique includes the steps of opening a sterilized packaging containing a disposable drive unit and/or attachment, and coupling the disposable drive unit and/or attachment to a surgical tool. The surgical technique additionally includes the step of performing a surgical procedure with the surgical tool. The surgical technique further includes the step of disposing the drive unit and/or attachment upon completion of the surgical procedure.

Further forms and embodiments of the present invention will become apparent from the detailed description provided hereinafter. It should be understood that the detailed description and specific examples, while indicating the preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is an environmental view of a surgical instrument for the dissection of bone and other tissue according to the teachings of a preferred embodiment of the present invention operatively associated with a patient for performing a craniotomy.

FIG. 2 is a side elevational view of the surgical instrument for the dissection of bone and other tissue according to the teachings of the preferred embodiment of the present invention, the surgical instrument shown operatively associated with a hose assembly.

FIG. 3 is an enlarged side elevational view of a drive unit of the surgical instrument of FIG. 2.

FIG. 4 is a cross-sectional view of the portion of the surgical instrument shown in FIG. 3.

FIG. 5 is a partially exploded side view of the drive unit of the surgical instrument shown in FIG. 3.

FIG. 6 is a partially exploded side view of the drive unit and attachment of the surgical instrument shown in FIG. 3.

FIG. 7 is a partially exploded side view of the attachment shown in FIG. 6.

FIG. 8 is a partial cross-sectional view of the attachment shown in FIG. 6.

FIGS. 9a and 9b are cross-sectional views of different embodiments of a motor for use in the surgical instrument shown in FIG. 3.

FIG. 10 is a cross-sectional view of one embodiment of the motor housing, taken from a view indicated in FIG. 9b.

FIG. 11 is a side elevational view similar to FIG. 2, illustrating a surgical instrument for the dissection of bone and other tissue according to the teachings of a first alternative embodiment of the present invention.

FIG. 12 is another side elevational view similar to FIG. 2, illustrating a surgical instrument for the dissection of bone and other tissue according to the teachings of a second alternative embodiment of the present invention.

FIGS. 13 and 14 are side elevational views of surgical instrument kits in accordance with the teachings of the present invention, the surgical instrument kits being pre-sterilized and packaged for immediate use in a surgical environment.

FIG. 15 is a side elevational view of a single-use coupling of the present invention for connecting a drive unit with a fluid hose.

FIGS. 16A-16D illustrate various exploded views of the single-use coupling of FIG. 9.

FIG. 17 is a cross-sectional view taken along the line 11-11 of FIG. 9, illustrating the single-use coupling in an assembled condition.

FIG. 18 is a cross-sectional view similar to FIG. 11, illustrating the single-use coupling of the present invention in a twisted condition immediately prior to decoupling.

FIG. 19 is a cross-sectional view of an electrical path modified so that a connected surgical instrument with an electrical motor is only used once.

FIGS. 20 and 21 are cross-sectional views of an air flow path modified according to different embodiments of the present invention so that the connected surgical instrument of FIGS. 2-5 is only used once.

FIG. 22 is a cross-sectional view of an air flow path modified so that the connected surgical instrument of FIGS. 2-5 is automatically lubricated.

### DETAILED DESCRIPTION OF THE PREFERRED EMBODIMENTS

The following description of the present invention is merely exemplary in nature and is in no way intended to limit the invention, its application, or uses.

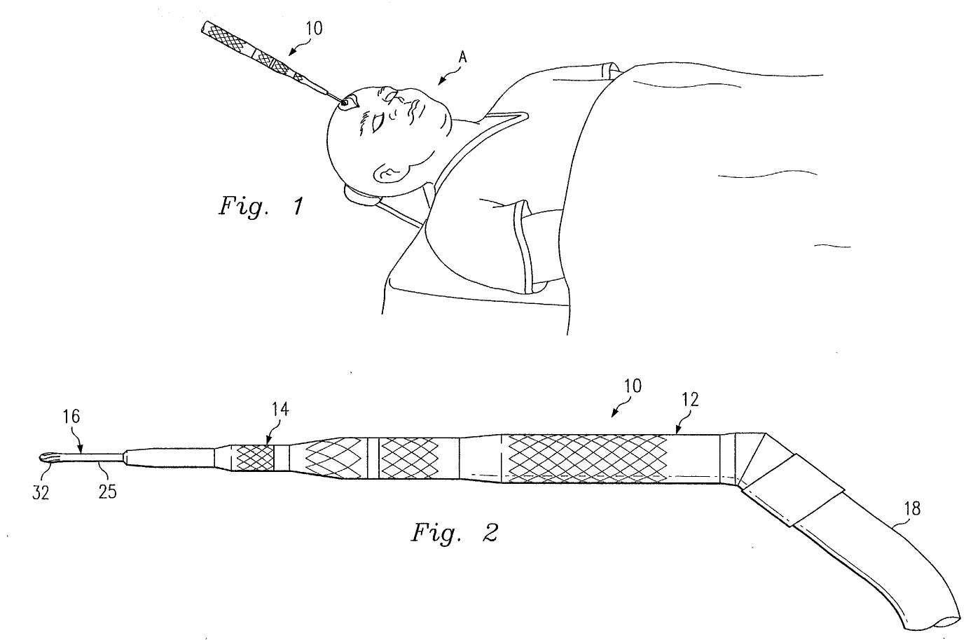

Referring initially to FIG. 1, a surgical instrument for the dissection of bone and other tissue constructed in accordance with the teachings of a first preferred embodiment of the present invention is illustrated and generally identified at reference numeral 10. The surgical instrument 10 is shown operatively associated with a patient A for performing a craniotomy. It will become apparent to those skilled in the art that the subject invention is not limited to any particular surgical application but has utility for various applications in which it is desired to dissect bone or other tissue. Additional applications include:

1. Arthroscopy—Orthopaedic

2. Endoscopic—Gastroenterology, Urology, Soft Tissue

3. Neurosurgery—Cranial, Spine, and Otology

4. Small Bone—Orthopaedic, Oral-Maxiofacial, Ortho-Spine, and Otology

5. Cardio Thoracic—Small Bone Sub-Segment

6. Large Bone—Total Joint and Trauma

7. Dental.

In the present disclosure, the surgical instrument 10 is disposable. As used herein, the term “disposable” describes something that can be used a relatively few number of times. For example, the disposable surgical instrument 10 may only be being operable for one, single procedure. Alternatively, the instrument may be used for multiple procedures with the same patient. Additional alternatives may also apply.

With reference to FIG. 2, the disposable surgical instrument lO is illustrated to generally include a handpiece or drive unit 12, an attachment 14, and a surgical tool 16. In the preferred embodiment, the surgical tool 16 is a cutting tool or dissection tool, although the type of tool is not essential to implementing the present invention. A distal end of the dissection tool 16 includes a cutting element.

The surgical instrument 10 is shown connected to a hose assembly 18 for providing a source of pressurized fluid (e.g., air) to the drive unit 12. As will become readily apparent to those skilled in the art below, the present disclosure is primarily directed to various features of a pre-sterilized and disposable surgical instrument. It is understood, however, that the teachings discussed herein may also apply to surgical instruments that can be reused and re-sterilized multiple times. In the exemplary embodiments that will be described, the surgical instrument 10 is pneumatically powered. It is further understood, however, that many of the teachings discussed herein will have equal application for an electrically powered surgical instrument.

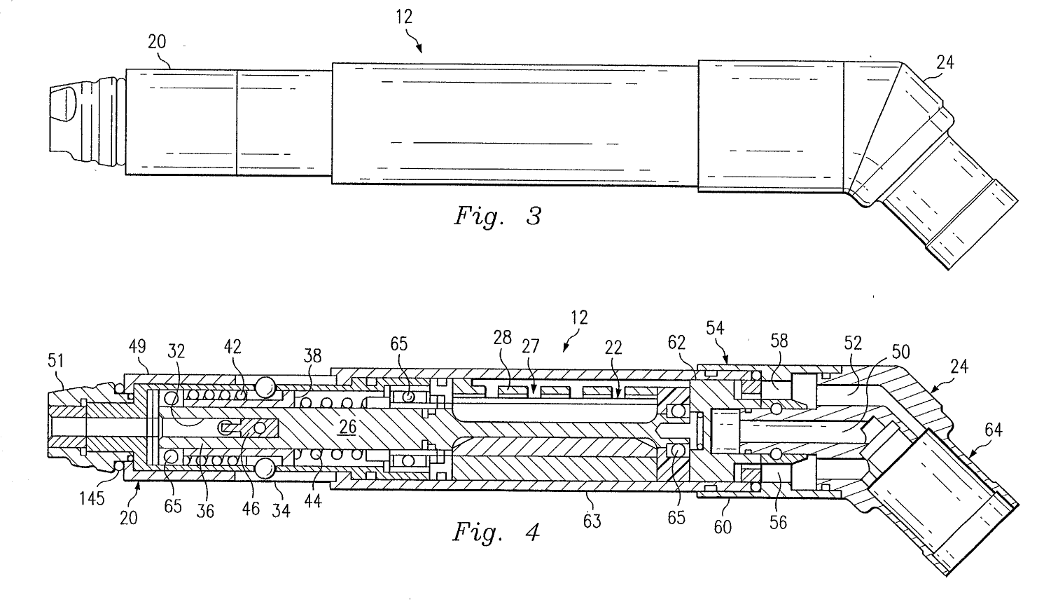

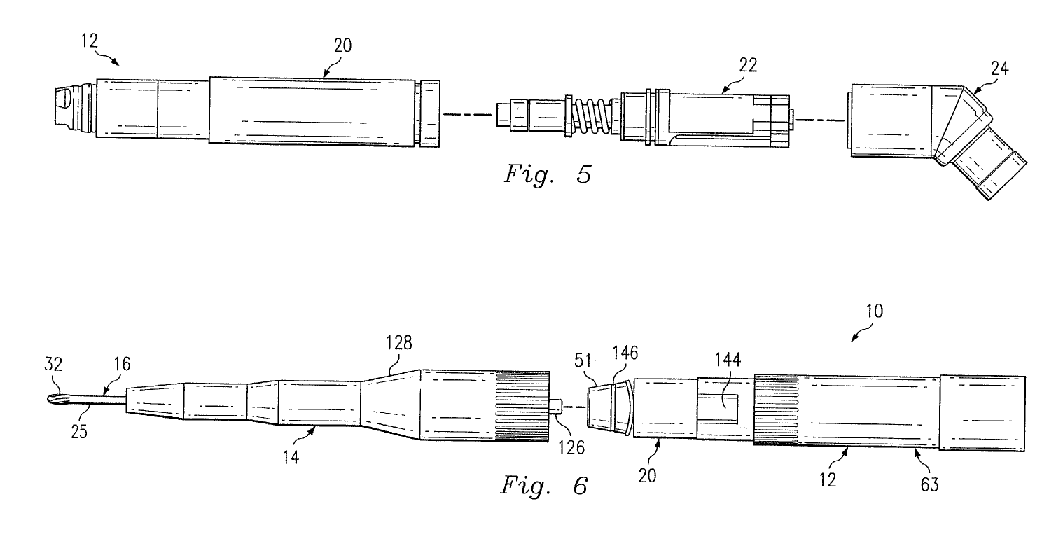

With continued reference to FIG. 2 and additional reference to FIGS. 3 through 5, the drive unit 12 of the present disclosure is shown to generally include a collet assembly 20, a motor assembly 22 and a hose connection assembly 24. The collet assembly 20 receives the dissection tool 16. The motor assembly 22 drives the dissection tool 16. The hose connection assembly 24 releasably interconnects the motor assembly 22 and the air hose 18.

In one application, most of the drive unit 12 is not constructed out of stainless steel. Instead materials such as plastic are used. Other examples of materials include ceramic, brass, aluminum, magnesium, or zinc. Manufacturing alternatives include, but are not limited to, die casting, investment casting, plastic injection molding and metal injection molding. Each of the possible materials advantageously reduces machining costs compared to conventional stainless steel and are suitable for disposable use. To some extent, each of these materials reduces instrument weight. Some materials, such as plastic, have the additional advantage of being radiotranslucent.

With particular reference to the cross-sectional view of FIG. 4, the collet assembly 20 will be described in further detail. The collet assembly 20 is illustrated to include a spindle 26 that engages and drives the dissection tool 16 about the axis of the spindle 26. The spindle 26 is driven by a rotor portion 27 of the motor assembly 22 through a plurality of rotor vanes 28. In one particular application, the plurality of rotor vanes includes four (4) rotor vanes 28. The rotor portion 27 is integrally formed with the spindle 26. The distal end of the spindle 26 defines a cylindrical cavity 32 for receiving the proximal end of the dissection tool 16.

The collet assembly 20 is further illustrated to include a rotatable member 34 and pads or locking members 36. In the particular embodiment illustrated, the surgical instrument 10 includes three locking members 36. An inner surface of each of the locking members 36 is operable to engage a reduced diameter groove (not shown) of the dissection tool 16. The outer surface of the locking members 36 is able to engage an axially translatable sleeve 38. Each of the locking members 36 is disposed within a radially extending aperture formed in the spindle 26 and intersecting the cavity 32. The locking members 36 are positioned and sized to be received within the reduced diameter portion of the dissection tool 16 when the dissection tool 16 is fully inserted into the cavity 32.

The sleeve 38 is generally tubular in shape and includes a central aperture for receiving the spindle 26. The sleeve 38 is axially movable axially along the spindle 26 between a first or rearward position and a second or forward position. In the first position (shown in FIG. 4), the sleeve 38 is axially displaced from the locking members 36 and the locking members 36 are free to move in a radially outward direction. In this first position of the sleeve 38, the dissection tool 16 may be withdrawn from the cavity 32 for quick and easy replacement. In the second position (not shown), the sleeve 38 maintains engagement of the locking members 36 with the reduced diameter portion of the dissection tool 16 thereby both preventing (1) inadvertent withdrawal of the dissection tool 16 from the surgical instrument 10 and (2) rotatably coupling the dissection tool 16 with the spindle 26.

Axial translation of the sleeve 38 is accomplished through manual rotation of the rotatable member 34 through approximately 90°. Explaining further, when the rotatable member 34 is rotated in a first direction, the sleeve 38 is forwardly translated against the spring bias of a first coil spring 42. Such forward translation radially positions the sleeve 38 over the locking member 36. The sleeve 38 is returned to its first or rearward position against the bias of a second coil spring 44 through rotation of the rotatable member 34 in a second direction.

The surgical instrument 10 further includes a pin 46 that extends from the spindle 26 into the cavity 32. When the dissection tool 16 is fully inserted into the cavity 32, the pin 46 extends into a bore of the dissection tool 16 thereby facilitating proper alignment of a central longitudinal axis of the dissection tool 16 with a rotational axis of the spindle 26.

The collet assembly 20 is shown to further include a fixed housing portion 49 and a nose portion 51. In one application, the fixed housing portion 49, the nose portion 51, the rotatable member 34 and the sleeve 38 are all constructed of a plastic material. Plastic advantageously reduces cost and weight. Additionally, plastic is inherently radiotranslucent. Such a characteristic may be important for surgical procedures requiring x-ray imaging. Further, all components may be formed of non-electrically conducting materials. An instrument formed of such materials has applications both in X-ray imaging and magnetic resonance imaging. While any variety of materials broadly referred to as plastics may be utilized herein, some specific examples include, but are not limited to, acetel, vespel (thremoset and thermoplastic), abs, polycarbonate, glass bead acetel and ultem. Alternatively, these plastic components may be manufactured of mild steel, aluminum, zinc, magnesium, brass or other suitable metals. Such materials are easier and less expensive to machine that traditional materials and are suitable (as is plastic) for the disposable surgical instrument 10 of the present disclosure since rusting and other longevity issues are not presented.

While the present illustrative embodiments show pneumatic powered motors, it is contemplated that the improvements described herein may be applied in an equal fashion to other motors, such as electric operating on AC or DC currents and ultrasonic motors run by piezo-electric or magneto-strictive forces. More specifically, components previously milled from metal may be formed of alterative materials including plastics and less expensive manufacturing techniques such as stamping, rolling, casting, etc. may be utilized to form metallic elements. Further, the durability of electrical connections may be downgraded in view of the disposability of the drive unit 12. It is contemplated that the pneumatic and electric motors, including associated components, may be designed to provide only a limited length of operational life.

The drive unit 12 is shown to further include a cylindrical housing 63 within which the rotor portion 27 and spindle 26 are rotatably supported. The housing 63 is preferably constructed of plastic. Alternatively, the housing 63 can be constructed of ceramic, brass, aluminum or mild steel. The advantages of each of these materials as compared to the stainless steel of conventional constructions are detailed elsewhere herein.

The combined rotor portion 27 and spindle 26 is rotatably supported within the drive unit 12 by a plurality of bearing assemblies 65. In one application, the bearing assemblies 65 are prelubricated. Due to the disposable nature of the surgical instrument 10, a reduced life for the instrument 10 is anticipated. Partially for this reason, conventional introduction of a lubricant carried by the source of pressurized air is not always required. Such prelubricated bearing assemblies 65 may be in the form of porous bearings (powdered or sintered metallurgy technologies) that are impregnated with lubricant. Additionally or alternatively, prelubrication can come from oil impregnated plastic material. Prelubricated bearing assemblies for other applications are well known. For example, prelubricated bearing assemblies are shown in U.S. Pat. Nos. 6,336,745, 6,270,259, 6,179,470, 5,834,870, and 5,120,140, which are hereby incorporated by reference as if reproduced herein in their entirety.

In the embodiment illustrated, the hose connection assembly 24 is an angled connection assembly and serves to releasably connect the drive unit 12 to the air hose assembly 18. The hose connection assembly 24 is illustrated to define a first fluid path 50 for delivering a source of pressurized air to the motor assembly 22 and a second fluid path 52 for returning a source of exhaust air. In some embodiments, the hose connection assembly 24 may include a plurality of segmented members that allow a user to change the position of the hose 18 in relation to the drive unit 12, and further help to reduce noise.

A first end 54 of the hose connection assembly 24 releasably receives an end of the motor assembly 22. As illustrated, an outlet port 56 of the connection assembly 24 is received within an inlet port 58 of the motor assembly 22. An outer cylindrical housing 60 of the connection assembly 24 receives a cylindrical end 62 of the drive unit 12.

A second end 64 of the hose connection assembly 24 releasably receives an end of the air hose assembly 18. While not particularly shown, it will be understood that the hose assembly 18 generally includes an outer conduit concentrically arranged with an inner conduit. The outer conduit defines a portion of the fluid path for transmitting exhaust gases away from the motor assembly 22 of the surgical instrument 10. The inner conduit defines a portion of the fluid path for transmitting the source of pressurized air to the motor assembly 22.

The surgical instrument 10 of the present disclosure is further shown to include the attachment 14. The attachment 14 rotatably supports the dissection tool 16 and protects tissue during a surgical procedure. In the exemplary embodiment, the attachment 14 is intended for a single-use and is disposable.

With continued reference to FIG. 6 and additional reference to FIG. 4, the drive unit 12 is shown to include a first portion and a second portion. The first portion 63 houses the motor assembly 22 and the second portion comprises the collet assembly 20 for releasably receiving a proximal end 126 of the dissection tool 16. As will be addressed below, the dissection tool 16 is axially retained within the collet assembly 20 through rotation of the collet assembly 20 relative to the first portion 63 of the drive unit 12. In the embodiment illustrated, the drive unit 12 includes a pneumatic motor and the first portion 63 of the motor is adapted to receive an air hose 127 in a conventional manner. Alternatively, it will be understood that the drive unit can include an electric motor powered through an electrical power cord or battery power.

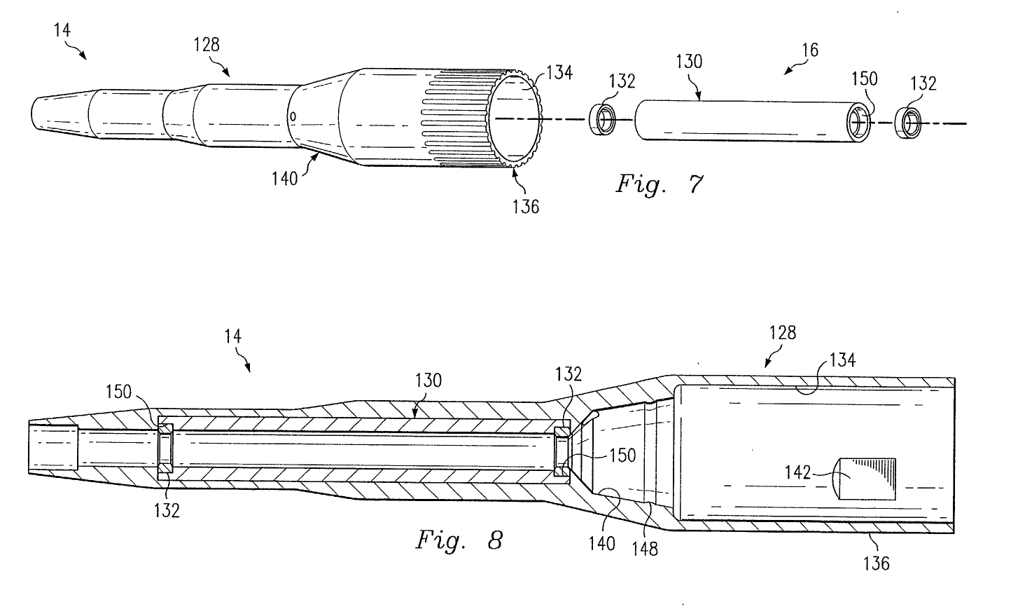

As most particular shown in the exploded view of FIG. 7 and the cross-sectional view of FIG. 8, the attachment 14 generally includes a main body portion 128, a tubular sleeve 130, and a pair of bearings 132. In the present embodiment, the bearings 132 are bushings. Because of their limited use, the attachment 14 can be constructed of materials that are easier and/or cheaper to manufacture and will not necessarily survive years of service and repeated sterilization processes. In one particular application, the main body portion 128 is a generally hollow member constructed of a disposable medical grade plastic such as polycarbonate. However, it will be understood that other materials, such as one or more of those materials listed throughout this specification and/or those having a low thermal conductivity, may be used.

The main body portion 128 includes a channel 134 extending along its axial length. At the proximal end 136 of the main body portion 128, the channel 134 is configured to matingly receive the collet assembly 20 of the drive unit 12. In this manner, the channel 134 includes a generally cylindrical section adjacent a tapered portion 140. The tapered portion 140 receives a nose portion 51 of the collet assembly 20 (FIG. 4).

Referring also to FIG. 8, when the main body portion 128 is removably attached to the drive unit 12, an inwardly extending flat portion 142 formed within the channel 134 aligns with a corresponding flat portion 144 disposed on the collet assembly 20. These mating surfaces 142 and 144 prevent relative rotation between the main body portion 128 and the collet assembly 20. A snap ring 145 (shown in FIG. 4) or O-ring is carried within a circumferentially extending groove 146 formed on the nose portion 51 of the collet assembly 20. This retaining member 145 engages a corresponding circumferentially extending groove 148 defined in the channel 134 to thereby retain the main body portion 128 axially relative to the drive unit 12. As shown in FIG. 2, the main body portion axially surrounds a substantial length of the dissection tool 16.

The tubular sleeve 130 is centrally disposed within the channel 134. The bearings 132 are located within counterbored portions 150 in respective ends of the tubular sleeve 130. The bearings 132 rotatably support the dissection tool 16 within the attachment 14.

In the exemplary embodiment, the main body portion 128 is molded over the tubular sleeve 130 and the bearings 132. Alternatively, the main body portion 128 can be constructed of two or more discrete portions that are bonded or otherwise similarly attached.

In one particular application, the tubular sleeve 130 is constructed of aluminum. Alternatively, the tubular sleeve 130 may be constructed of stainless steel or other well known materials having a relatively high thermal conductivity and low density. The bearings 132 are preferably constructed of a molded polymer and graphite filled. It is understood that the term bearings can generically refer to bushings, ball bearings, air channels, ceramic journal bearings, and the like.

In use, a surgeon will grasp the attachment in a manner similar to a pencil. The sleeve 130 provides strength and rigidity to the attachment 14. The fairly high thermal conductivity of the sleeve and its low density causes the sleeve 130 to act as a heat sink and draw heat generated through friction away from the bearings 132 and the plastic main body portion 128. As a result, high temperatures are not transferred to the surgeon or soft tissue.

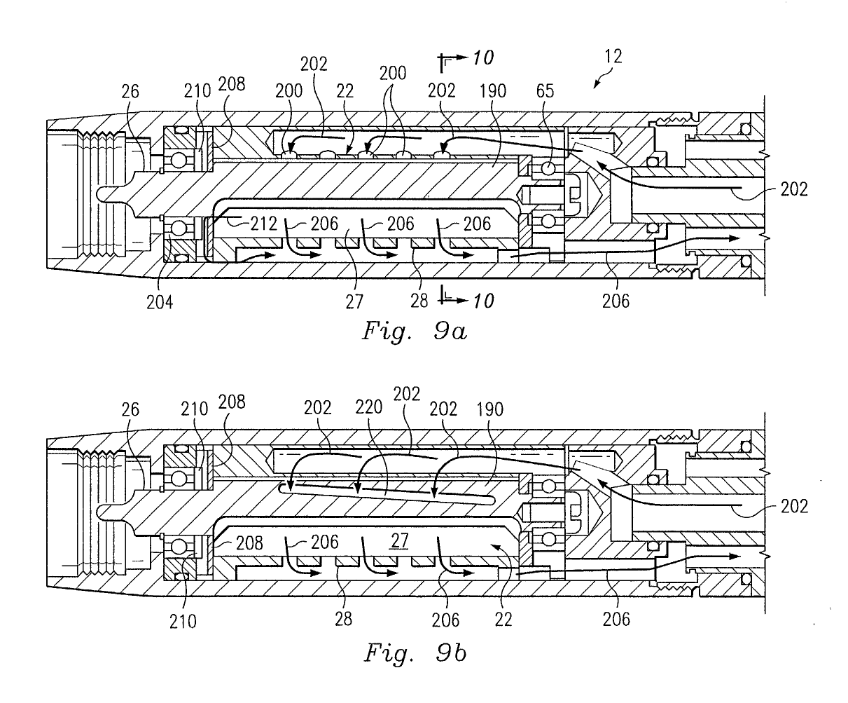

Referring to FIG. 9a, in one embodiment, the motor assembly 22 includes a vane housing 190 having a plurality of apertures 200 for receiving a high pressure fluid 202. The vane housing 190 and the apertures 200 can be made by conventional machining processes. In operation, the high pressure fluid 202 moves through the rotor portion 27. The moving fluid 202 causes the vanes 28 to rotate the spindle 26 on the bearing assemblies 65, 204. The high pressure fluid 202 escapes through as exhaust 206. In addition, a lubrication seal housing 208 and a lubrication seal 210 can direct any leaking fluid 202 (and/or oil that is included with the fluid) through a path 212 to rejoin the exhaust fluid 206. In this way, fluid 202 and/or oil is not projected towards the dissection tool 16 and the patient (FIGS. 1 and 2).

Referring to FIG. 9b, in another embodiment, the motor assembly 22 is similar to the one shown in FIG. 9a. However, instead of the vane housing 190 having a plurality of apertures 200, a slot 220 is provided for receiving the high pressure fluid 202. The vane housing 190 and the slot 220 can be made by conventional machining processes as well as die cast processes. An advantage of having the slot 220 is that it is easy to manufacture while still maintaining certain opening-size requirements.

Referring to FIG. 10, in some embodiments, the cylindrical housing 63 of the drive unit 12 may include a plurality of ridges 240 running longitudinally along the inside diameter of the housing, parallel with the spindle 26. The ridges 240 can collect a portion of the fluid used to operate the pneumatic motor assembly 22, thereby forming a “fluid ring” 242 that is concentric with the spindle 26. As a result, the fluid ring 242 acts as an insulator for both noise and heat. It is understood that other arrangements of ridges and protrusions can be used to produce similar effects.

In another embodiment, the cylindrical housing 63 can include an additional outer housing 244. The outer housing 244 helps to insulate noise and/or heat. In addition, the outer housing 244 can be made of a different material that is more desirable to being held by the surgeon holding the instrument 10. For example, the outer housing 244 can be made of a metal (like conventional instruments) or a softer padded material.

Furthermore, the cylindrical housing 63 can be manufactured with an increased inside diameter at one end to allow the loading of all the motor assembly 22 components from that one end during manufacturing. This can be easily done when the cylindrical housing 63 is made via a molding process (vs. a machining process).

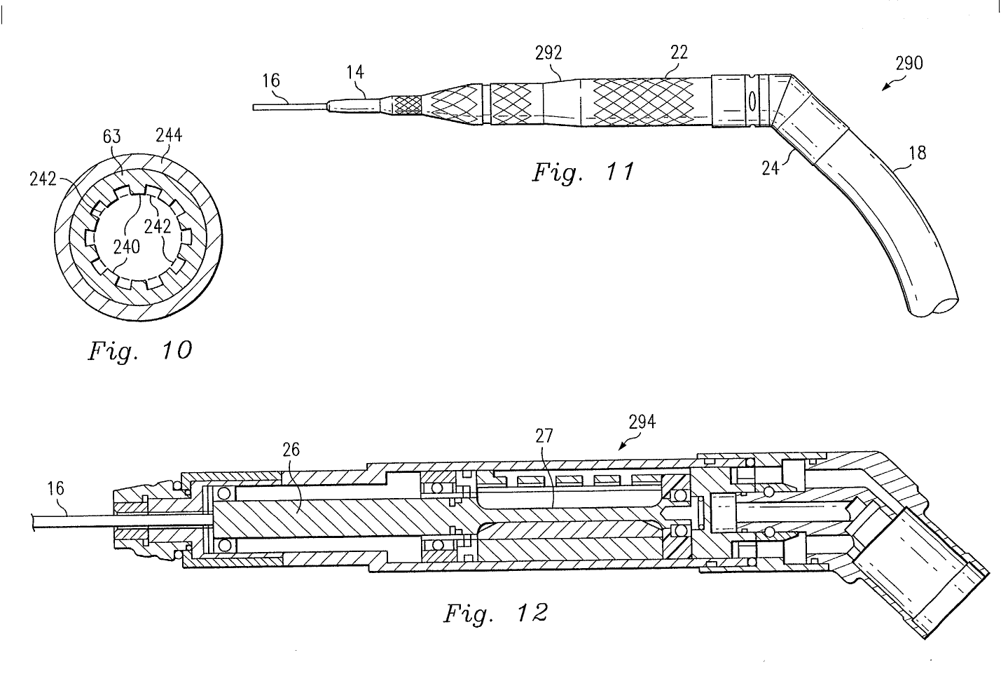

Turning now to FIG. 11, a surgical instrument for the dissection of bone and other tissue according to the teachings of an alternative embodiment of the present disclosure is shown in cross-section and generally identified at reference numeral 290. The surgical instrument 290 of the first alternative embodiment is similar to the surgical instrument 10 of the preferred embodiment. For this reason, like reference numerals are used to identify substantially identical components between the two embodiments. The surgical instrument 290 differs from the earlier described embodiment in that it incorporates a common housing 292 for both the attachment 14 and the drive unit 12. The common housing 292 may be constructed of plastic, steel, aluminum, bronze or other suitable materials. In some embodiments, the collet member is similar to the collet assembly 20 of FIG. 4, except that the dissection tool 16 cannot be released from the collet once attached.

Referring also to FIG. 12, a surgical instrument for the dissection of bone and other tissue according to the teachings of another alternative embodiment of the present disclosure is shown in cross-section and generally identified at reference numeral 294. In these embodiment, the dissection tool 16 is permanently secured to the spindle 26, and the dissection tool, the rotor portion 27, and the spindle may be integrally formed. As a result, the collet assembly 20 can be simplified. In these embodiments, the surgical tool 294 is intended for a procedure in which changing of the dissection tool 16 due to wear or varying cutting/dissection requirements is not needed. One such surgical procedure would include a craniotomy.

With reference to FIG. 13, a surgical instrument kit in accordance with the teachings of the present disclosure is illustrated and generally identified at reference number 300. The surgical instrument kit 300 includes various components that are pre-sterilized and packaged for immediate use in a surgical environment. The various components of the exemplary kit 300 are shown to include a drive unit 12 (including the disposable motor assembly 22), a plurality of attachments 14, and a plurality of dissection tools 16. The particular components 12, 14 and 16 of the illustrated surgical instrument kit 300 will be understood to be merely exemplary. In this regard, other arrangements of components can be selected for inclusion in a kit based on the intended surgical use of the particular kit.

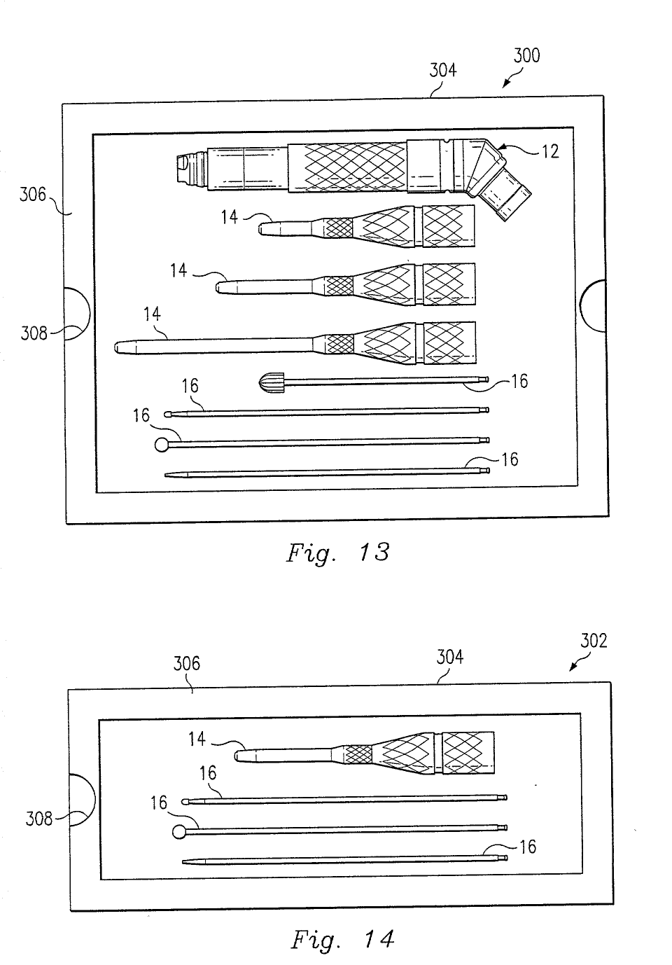

With reference to FIG. 14, according to a preferred method of the present invention, a kit 302 may include one or more attachments 14 and one or more dissection tools 16. The attachment 14 is offered as a pre-sterilized product. Preferably, the attachment 14 is capable of withstanding gamma irradiation and ethylene oxide sterilization. The attachment 14 may be intra-operatively removed from packaging and releasably attached to the drive unit 12. Opening and using a sterile attachment with each surgery theoretically reduces the risk of patient infection. After use of the surgical instrument 10 to dissect bone or other tissue, the attachment is removed from the drive unit 12 and discarded. Special handling for patients with infectious diseases such as AIDS and hepatitis is eliminated.

The surgical kits 300, 302 further includes a plastic package 304 used to envelope the components of the kit prior to its their use. The plastic package 304 includes a sealed edge 306 and opening tab 308. The sealed package 304 maintains sterility of the components of the kit 300 prior to the surgical procedure. Since the package may be assembled at a separate location, a very high level of sterility can be achieved.

According to a preferred method of the present invention, the surgical instrument kit 300 is offered as a pre-sterilized product. Preferably, each of the components of the kit 300 is capable of withstanding gamma irradiation or ethylene oxide sterilization. The components of the surgical instrument kit 300 are intended to be intra-operatively removed from the package 304. Opening and using sterile components with each surgery theoretically reduces the risk of patient infection.

Upon intra-operative removal of the components from the package 302, the drive unit 12 is releasably attached to a power source. In the exemplary embodiment in which the drive unit 12 is pneumatically driven, the drive unit 12 is releasably attached to a hose assembly 18 for the delivery of a source of pressurized air. After use of the surgical instrument 10 to dissect bone or other tissue, the drive unit 12, attachments 14 and dissection tools 16 are decoupled from the hose assembly 18 and discarded along with the package 304. Special handling of re-useable instruments for patients with infectious diseases such as AIDS and hepatitis is eliminated.

It will be understood that for certain applications it may be desirable to permanently secure the hose assembly 18 to the surgical instrument 10 in any manner well known in the art. In such an application, the dissection tool can be replaceable (as with the embodiment shown in FIG. 2) or not replaceable (as with the embodiment of FIG. 12). Also in such an application, the handpiece 12 can be formed integrally with the attachment 14 or can be detachably secured to the attachment. Still further, it will be recognized that the handpiece 12 and hose assembly 18 may be packaged in a kit and provided to the use in a sterilized condition.

In application where the hose assembly 18 is permanently secured to the handpiece 12, the hose 18 is preferably constructed of a low cost plastic material or a nonwoven material such as polyethylene coated with an interior liner material. One suitable laminated, non-woven material is commercially available under the registered trademark Tyvek. Alternatively, other non-woven materials having suitable tear strength and shear properties without the cumbersome bulk and weight characteristic of conventional silicone may be employed. A coupling assembly as described herein may be attached to the hose for connection to a pressurized air source.

In certain applications, it may be desirable to incorporate such a single-use coupling to prevent re-use of the surgical instrument 10 which may lead to infection or unacceptable degradation of the surgical instrument resulting from a sterilization procedure. The single-use coupling (replacing the coupling assembly 24) may be operative for connecting the motor assembly 22 with the air hose 18. In one embodiment, the single-use coupling may include a shear-able component that only allows a single connection. For example, when the coupling is disconnected, the component is sheared or otherwise destroyed. In the preferred embodiment, the shear-able component would be incorporated with the motor assembly 22.

Referring now to FIGS. 15 through 18, in another embodiment, a single-use coupling 400 includes a first portion or component 402 for attachment to the motor assembly 22 of the drive unit 12 and a second portion or component 404 for attachment to the air hose assembly 18. As will be come apparent below, the first and second components 402 and 404 are designed to quickly and easily couple so as to define fluid paths between the drive unit 12 and the hose assembly 18 and further so as to effectively destroy a retention mechanism upon decoupling to prevent reattachment.

The first component 402 includes one or more retainers for securing the first component 402 to the second component 404. In the particular embodiment illustrated, the first component 402 includes a pair of cantilevered legs 406 that extend in an axial direction. Each of the cantilevered legs 406 carries a radially extending tab or button 408. The legs 406 are radially opposed from one another.

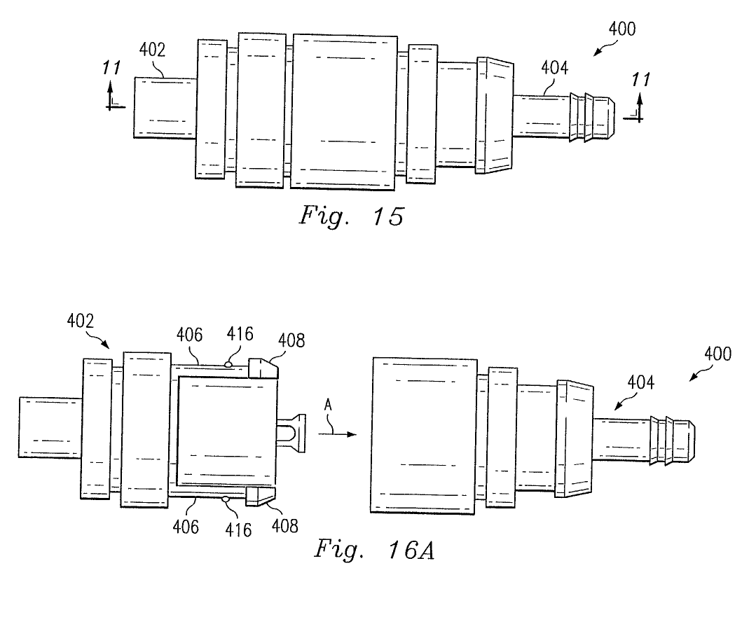

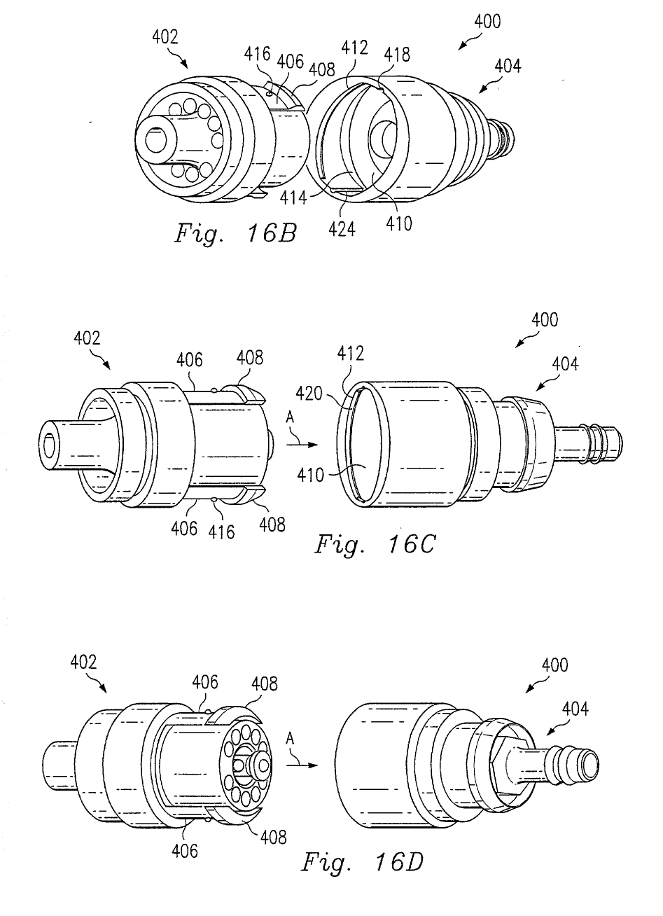

The legs 406 are integrally formed with a housing of the first component 402 of a plastic material and are resiliently deflected upon insertion in a generally cylindrical cavity 410 defined by the second component 404. Insertion into the cavity 410 is generally in the direction of arrow A (see FIGS. 16A-16C). An opening 412 to the cavity 410 is generally oval shaped and requires the legs 406 to be aligned along its long axis upon insertion. The buttons 408 are received behind a lip 414 (as shown in FIGS. 16B and 18) within the cavity 410. The lip 414 prevents withdrawal of the first component 402 from the second component 404 in a direction opposite to arrow A.

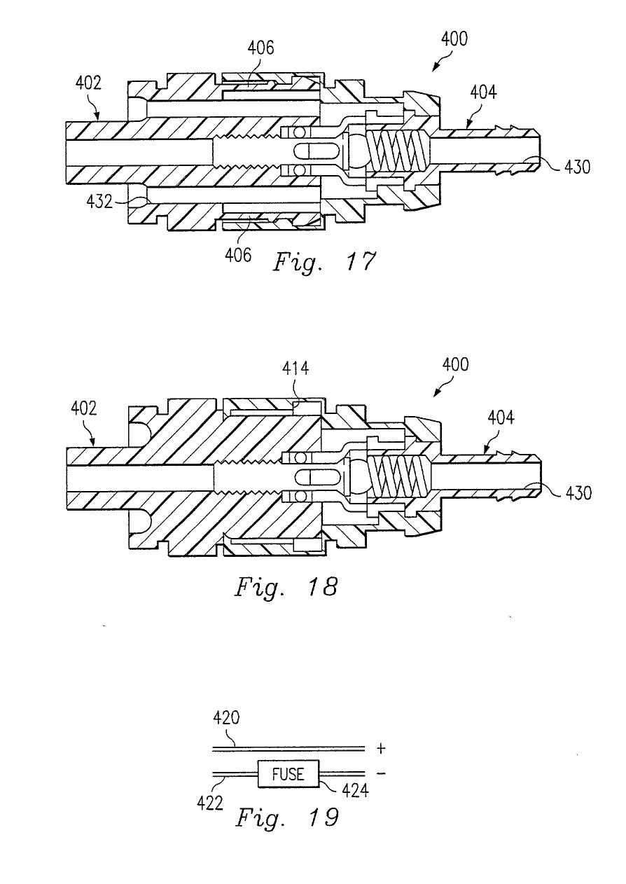

The second component 404 includes a pair of cutting members 420 (see FIG. 16C) for the destruction of the cantilevered legs 406. As used herein, the term “destruction” shall refer to an action that permits removal of the first component 402 from the second component 404 and effectively prevents reattachment of the first and second components 402 and 404. The cutting members comprise blades 420 positioned at laterally opposed sides of the opening 412. When it is desired to decouple the first and second components 402 and 404, the components 402 and 404 are relatively rotated from their assembly condition (as shown in FIG. 17) to a twisted position (as shown in FIG. 18). In the embodiment illustrated, the first component 402 is rotated either clockwise or counterclockwise relative to the second component 404 through approximately 90 degrees. This action causes the blades 420 to cut the legs 406 and thereby permit withdrawal of the first component 402.

Each of the legs 406 is illustrated to carry one or more partially spherical bumps 416. The bumps 416 align with axially extending grooves 418 defined by the housing of the second component 404. In this manner, inadvertent twisting of the first component 402 is prevented. Explaining further, intentional twisting must first resiliently deflect the legs 406 sufficiently enough to displace the bumps 416 from the grooves 418.

In some embodiments, the disposable powered surgical drive unit 12 and/or attachment 14 may purposely include one or more failure points so that they cannot be reused. This can be important to prevent the improper reuse of the device(s) for subsequent surgical procedures. For example, the drive unit 12 can include a component that adversely reacts to a high-temperature autoclave or other sterilization process.

Referring now to FIG. 19, in one embodiment with an electrical motor 22, one or both electrical supply leads 420, 422 which are used to supply power to the motor from a power source (not shown) may include a temperature-sensitive fuse 424. The fuse 424 electrically breaks when exposed to a moderately high temperature. Continuing with the example above using the autoclave, the break point of the fuse 424 can be about 200° C. In this way, once the substance fuse 424 is subject to an autoclave or other high-temperature sterility process, electrical connection to the motor is permanently interrupted. Other fuses may be used that react to different sterilization processes.

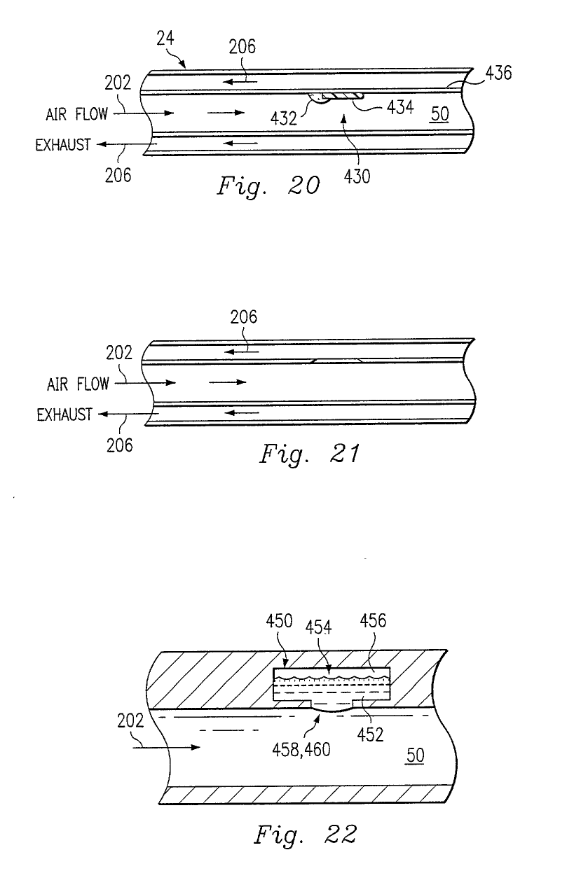

Referring now to FIG. 20, in another embodiment, the hose connection assembly 24 includes a flow interrupter 430 in the first fluid path 50. The flow interrupter 430 includes a low-melting point substance 432 (and in some embodiments a fluid obstruction device 434) positioned within the instrument, such as being attached to a surface of the structure 436 defining the first fluid path. In a preferred embodiment, the flow interrupter 430 is positioned at a distance from the drive unit 12 (FIG. 2) to reduce any affects from natural heat generation created by the motor and surrounding components during operation.

The low-melting point substance 432, which converts to a liquid state when exposed to a moderately high temperature. By way of example but without limitation, such materials may include waxes and plastics, such as biodegradable and starch based polymers. For the sake of further example, since autoclave temperatures are typically about 270° C., the melting point of the substance 432 can be about 200° C. In this way, if the substance 432 is ever subject to an autoclave or other high-temperature sterility process, it liquefies. In the present example, the fluid obstruction device 434 is released, rendering the pneumatic motor 22 inoperable. In other examples, the substance 432 alone or in combination with other components can serve to render the instrument in general inoperable.

Referring to FIG. 21, in another embodiment, the hose connection assembly 24 includes a meltable wedge 440 between the supply fluid path 50 and an exhaust fluid path 442. The wedge includes a portion that has a low-melting point, such as a wax-type substance or ethylene vinyl acetate (EVA). In a preferred embodiment, the wedge 440 is positioned at a distance from the drive unit 12 (FIG. 2) to reduce any affects from natural heat generation created by the motor and surrounding components during operation. If the wedge 440 is ever subject to an autoclave or other high-temperature sterility process, it liquefies. This creates a short between the supply fluid path 50 and the exhaust fluid path 442, so that at least a portion of the supply fluid 202 flows directly into the exhaust fluid 206.

In some embodiments, the drive unit 12 and/or attachment 14 may not require any type of liquid lubrication. Such embodiments may include components, such as the bearings 132 (FIG. 7) or the bearings 65, 204 (FIGS. 9a, 9b), formed of or coated with materials having a low coefficient of friction. Some of the components, such as the vanes 28, may also degrade with use. Through degradation, the components provide lubrication to the remaining portions of the instrument. In another example, porous bearing assemblies (e.g., ball bearing assemblies that use powdered or sintered metallurgy technologies) that are impregnated with lubricant can be used. In another example, a thixotropic gel can be used to provide lubrication. In addition, the change of phase (from a solid to a liquid) of the thixotropic gel during operation of the instrument 10 also serves to absorb heat.

Referring now to FIG. 22, in some embodiments, an internal lubrication system can be provided so that external lubrication does not need to be provided. The lubrication system can be self-contained, self-metering, and self-initializing. For example, a self-lubricating system 450 can be incorporated into the first fluid path 50. The self-lubricating system 450 includes a lubricating fluid (e.g., oil) stored in a reservoir 454. Additional air 456 may be included in the reservoir 454 for reasons discussed below. In the embodiment shown in FIG. 15, reservoir 454 is sized to contain enough lubricating fluid to accommodate the use of drive unit 12 for a limited period of time. Thus, the embodiment of FIG. 15 is particularly well suited to be incorporated into a disposable drive unit 12 or single-use air supply hose as disclosed herein. However, it is contemplated that the oil reservoir may be larger than shown to accommodate multiple-use applications. Further, it is contemplated that the embodiment of FIG. 15 may be a disposable component that may be coupled to an air supply hose for lubricating a single-use or multiple-use pneumatic motor. An alternative lubricating system that may have application in combination with the present invention is disclosed in commonly assigned U.S. Ser. No. 60/301,491, incorporated herein by reference.

The reservoir 454 includes an orifice 458 covered by a membrane 460. When pressurized air is provided in the fluid path 50, membrane 460 moves substantially in the direction indicated by arrow 202 as a result of the compression of air 456. The pressurized air may be about 100-22 pounds per square inch (psi). With this pressure, the pressurized air is capable of providing sufficient force to rupture the membrane 460. This rupturing can be facilitated by the air 456 in the reservoir 454, which may be compressed to accommodate movement in the membrane 460. Once ruptured, the oil 452 is released into the first fluid path 50, where it is atomized and combined with the pressurized air. The atomized oil can then be used to lubricate the surgical instrument in a conventional manner.

The orifice 458 is of a sufficient size so that the rate at which the oil 452 is atomized is controlled. In this way, the lubrication is self-metered. When there is no air movement in the fluid path 50, capillary forces of the oil 452 will sustain the oil in the reservoir. In this way, the surgical instrument will be lubricated for a predefined period of operation, after which the lack of lubrication will eventually cause the drive unit to fail.

The description of the invention is merely exemplary in nature and, thus, variations that do not depart from the gist of the invention are intended to be within the scope of the invention. For example, some of the lubrication mechanisms or failure mechanisms may actually survive more than one use and/or sterilization process. Also, different combinations of the materials and manufacturing methods discussed above can be used on the various components of the surgical instrument 10. In this way, factors such as cost, translucence, weight, heat conduction, vibration, look and feel, reliability, and strength can be balanced for a particular process or application. Such variations are not to be regarded as a departure from the spirit and scope of the invention.

What is claimed is:

1. A surgical instrument for the dissection of bone and other tissue, comprising:
a powered drive unit made substantially of non-metal material; and
a dissection tool operably connected to the drive unit.

2. The surgical instrument of claim 1 wherein the drive unit includes a pneumatic motor made substantially of plastic.

3. The surgical instrument of claim 1 wherein the dissection tool is a rotary-type tool, the instrument further comprising:
an attachment connected to the drive unit and surrounding the dissection tool.

4. The surgical instrument of claim 3 wherein the attachment is made substantially of non-machined material and wherein the drive unit and the attachment are manufactured to survive a single operating procedure.

5. A drive unit for use with a rotary-type surgical instrument, the drive unit comprising:
a first interface for connecting to a power source;
a second interface for connecting to a surgical tool;
a motor connected to the first and second interfaces; and
means for making the drive unit disposable.

6. The drive unit of claim 5 wherein the means for making the drive unit disposable degrades the motor in response to being exposed to an sterilization process.

7. The drive unit of claim 5 wherein the means for making the drive unit disposable disables the drive unit in response to being exposed to an autoclave process.

8. The drive unit of claim 5 wherein the means for making the drive unit disposable provides a predetermined amount of lubricant to be supplied to the motor.

9. The drive unit of claim 5 wherein the means for making the drive unit disposable is a rapidly-degradable material used in the motor.

10. The drive unit of claim 5 wherein the means for making the drive unit disposable disables the drive unit in response to disconnecting the first interface from the power source.

11. The drive unit of claim 5 wherein the means for making the drive unit disposable includes a single-use coupling for use with the first interface.

12. The drive unit of claim 5 wherein the means for making the drive unit disposable limits an amount of power that can be provided from the power source to the motor.

13. A surgical instrument comprising:
a surgical tool; and
a drive unit having a rotatable motor, the rotatable motor being permanently secured to the surgical tool.

14. The surgical instrument of claim 13 wherein the drive unit is made substantially of moldable material.

15. A surgical instrument for the dissection of bone and other tissue, the surgical instrument comprising:
a rotary cutting member;
a motor assembly for driving the rotary cutting member including a collet assembly for receiving the rotary cutting member; and
an attachment including a hollow main body portion circumferentially surrounding a substantial portion of the rotary cutting member, a tubular sleeve disposed in the main body portion and a pair of bearings disposed in the tubular sleeve, the pair of bearings rotatably supporting the rotary cutting member, the main body portion releasably attached to the motor assembly.

16. The surgical instrument of claim 15, wherein the main body portion is constructed of a first material having a low thermal conductivity.

17. The surgical instrument of claim 16, wherein the tubular sleeve is constructed of a second material having a high thermal conductivity.

18. A pneumatic drive unit for use with a surgical instrument, comprising:
a rotor positioned around a rotational axis of the drive unit; and
a vane housing surrounding the rotor and having an elongated slot extending in a direction of the rotational axis at a radial distance from the axis;
wherein the slot is for directing a fluid towards the rotor for turning the rotor about the rotational axis.

19. A pneumatic drive unit for use with a surgical instrument, comprising:
a motor; and
a housing surrounding the motor, the housing including a plurality of ridges;
wherein the housing is operable for receiving a fluid and directing a first portion of the fluid to the motor, and at least temporarily sustaining a second portion of the fluid between the plurality of ridges.

20. A drive unit for use with a surgical instrument, comprising:
a motor; and
a first housing surrounding the motor, the first housing being made of a first material;
a second housing surrounding the first housing being made of a second material.

21. The drive unit of claim 20 wherein the second material is an over-molded piece of plastic.

22. A surgical instrument comprising:
a powered drive unit made substantially of radio-translucent material; and
a dissection tool operably connected to the drive unit.

23. A surgical instrument comprising:
a pre-sterilized drive unit made substantially of extrudable or moldable material; and
a container for maintaining the sterility of the drive unit until the drive unit is ready to be used.

24. A single-use coupling for connecting a fluid hose with a surgical instrument having a pneumatically powered motor, the single-use coupling comprising:
a first portion interconnected to the pneumatically powered motor; and
a second portion interconnected to the fluid hose;
wherein the first and second portions are selectively couple-able to define a fluid path between the pneumatically powered motor and the fluid hose; and
wherein one of the first and second portions includes at least one retainer for securing the first portion to the second portion when coupled, and the other of the first and second portions includes a cutting member for destruction of the at least one retainer upon decoupling of the first and second portions.

25. A kit for a surgical procedure, the kit comprising:
a motor for coupling to a source of power;
at least one dissection tool to be driven by the motor unit; and
a sealed package enveloping the motor and the at least one dissection tool;
wherein the sealed package maintains sterility of the motor and the at least one dissection tool prior to the surgical procedure.

26. A kit for a surgical procedure, the kit comprising:
an attachment for coupling to a motor;
a dissection tool to be driven by the motor unit and positioned inside the attachment; and
a sealed package enveloping the attachment and the dissection tool;
wherein the sealed package maintains sterility of the attachment and the dissection tool prior to the surgical procedure.

27. A method of using a surgical instrument having a rotary cutting member driven by a motor assembly to dissect bone or other tissue, the method comprising the steps of:
providing a pre-sterilized attachment for circumferentially surrounding a substantial length of the rotary cutting member;
intra-operatively removing the attachment from a sealed package;
removably attaching the attachment to the motor assembly such that the attachment circumferentially surrounds a substantial length of the rotary cutting member;
using the surgical instrument to dissect bone or other tissue;
removing the attachment from the motor portion; and
discarding the attachment.

28. A method of using a surgical instrument, the method comprising the steps of:
providing a pre-sterilized drive unit for providing a rotational force to a dissection tool;
intra-operatively removing the attachment from a sealed package;
attaching the drive unit so the dissection tool to construct the surgical instrument;
using the surgical instrument to dissect bone or other tissue; and
discarding the drive unit.

29. A method of providing a surgical instrument, the method comprising the steps of:
manufacturing a disposable motor for use with the surgical instrument;
providing the disposable motor to an end user for performing a surgical operation; and
receiving the disposable motor from the end user upon completion of the surgical operation.

30. The method of claim 29 further comprising:
crediting the end user upon receipt of the disposable motor.

31. The method of claim 29 wherein the disposable motor is received at a facility that is different from a facility used for manufacturing the disposable motor.
